(19) United States
(12) Patent Application Publication
(10) Pub. No.: US 2025/0213440 A1
(43) Pub. Date: Jul. 3, 2025

(54) GEL HAND SANITIZER AND PREPARATION METHOD THEREFOR

(71) Applicant: Solventum Intellectual Properties Company, Maplewood, MN (US)

(72) Inventors: Xin Yu, Shanghai (CN); Zhilan Liu, Shanghai (CN); Fang yuan Hu, Shandong (CN); Xi Zhang, Shanghai (CN)

(21) Appl. No.: 18/852,590

(22) PCT Filed: Mar. 31, 2022
(86) PCT No.: PCT/CN2022/084278

§ 371 Date: Sep. 30, 2024

(51) Int. Cl. A61K 8/22; A61K 8/04; A61K 8/34; A61K 8/41; A61K 8/81; A61Q 17/00

(52) U.S. Cl. CPC A61K 8/22; A61K 8/042; A61K 8/34; A61K 8/345; A61K 8/41; A61K 8/8147; A61Q 17/005; A61K 2800/48

(57) ABSTRACT

Provided in the present invention are a gel hand sanitizer and a method for preparing a gel hand sanitizer. The gel hand sanitizer comprises, based on the total weight thereof: 75-85 wt % of a monohydric alcohol; 0.05-1 wt % of hydrogen peroxide; 0.1-2 wt % of a thickener; 0.01-1 wt % of a neutralizer; and the balance of water. The neutralizer has a structure represented by the general formula: CₐH_{b}N_{c}O_{d}, where a≥6, b≥15, 1≤c<3, and 1≤d<6. According to the technical solution of the present invention, a gel hand sanitizer with good stability, moisture retention, and good fungi and virus killing effects can be provided.

### TECHNICAL FIELD

The present invention relates to the technical field of cleaning. In particular, provided in the present invention are a gel hand sanitizer and a preparation method therefor.

### BACKGROUND

Hand washing is an important part of maintaining personal hygiene in daily life. At the same time, hand washing is also an operation that hospital medical staff need to repeat frequently, which is essential to prevent or reduce cross-infections of related diseases in hospitals. There are currently various hand sanitizer products on the market, such as liquid hand sanitizers, foam hand sanitizers, and gel hand sanitizers. The gel hand sanitizers are more convenient to use than other forms of hand sanitizers, have stable dispense volume, and do not splash and flow easily. At present, requirements for gel hand sanitizers are getting higher. In addition to the ability to provide good cleaning effects, they are also required to have good stability properties, moisture retention properties, fungi killing effects, and virus killing effects.

Therefore, it is of great significance to develop a gel hand sanitizer with good stability, moisture retention, and good fungi and virus killing effects.

### SUMMARY

Starting from the technical problem elaborated above, a purpose of the present invention is to provide a gel hand sanitizer with good stability, moisture retention, and good fungi and virus killing effects, and a preparation method therefor. The inventors of the present invention have conducted intensive and detailed research to obtain the present invention.

According to one aspect of the present invention, provided is a gel hand sanitizer, comprising, based on the total weight thereof:
75-85 wt % of a monohydric alcohol;
0.05-1 wt % of hydrogen peroxide;
0.1-2 wt % of a thickener;
0.01-1 wt % of a neutralizer; and
the balance of water;
wherein the neutralizer has a structure represented by the general formula:

CₐH_{b}N_{c}O_{d}, where a≥6, b≥15, 1≤c<3, and 1≤d<6.

According to another aspect of the present invention, provided is a method for preparing a gel hand sanitizer, comprising mixing the components of the gel hand sanitizer described above.

The gel hand sanitizer according to the present invention has good stability and moisture retention, and at the same time has effective killing effects on both fungi and viruses.

### DETAILED DESCRIPTION

It should be appreciated that various other embodiments could be devised and modified by a person skilled in the art in light of the teachings of this description without departing from the scope or spirit of the present disclosure. Therefore, the following embodiments are not restrictive in meaning.

Unless otherwise indicated, all numbers used in this description and claims for the dimensions, quantities, and physicochemical properties of features should be construed to be modified by the term “approximately” in all instances. Accordingly, unless indicated to the contrary, the above numerical parameters listed in the description and attached claims are all approximations, which can be properly altered by a person skilled in the art using desired properties sought to be obtained from the teachings disclosed herein. The use of numerical ranges indicated by endpoints includes all numbers within that range and any range within that range, for example, 1 to 5 includes 1, 1.1, 1.3, 1.5, 2, 2.75, 3, 3.80, 4, and 5, etc.

At present, with the development and application of hand sanitizer products, technical requirements for the hand sanitizer products are increasingly high. It is desired that the hand sanitizer products have good stability, moisture retention, and effective killing effects on common fungi and viruses in addition to good cleaning effects. Currently, the most common commercial gel hand sanitizer products typically contain components such as ethanol, thickeners, and moisturizers. The inventors of the present invention found that these commercial gel hand sanitizer products containing only the aforementioned components are not satisfactory in terms of fungi and virus killing effects. For example, although these products have a certain killing effect on Escherichia coli, Staphylococcus aureus, and Pseudomonas aeruginosa, they have no obvious killing effects on Candida albicans, which is common in daily life, and has poor killing effects on polioviruses. The inventors of the present invention found in research that by using a combination of specific contents of a monohydric alcohol, hydrogen peroxide, a specific thickener, a neutralizer, and water, it is possible to provide a hand sanitizer which has good stability and moisture retention, and also good killing effects on a broad range of common fungi and viruses (including Candida albicans and polioviruses).

### Gel Hand Sanitizer

In certain aspects of the present invention, provided is a gel hand sanitizer. The gel hand sanitizer includes, based on the total weight thereof.
75-85 wt % of a monohydric alcohol;
0.05-1 wt % of hydrogen peroxide;
0.1-2 wt % of a thickener;
0.01-1 wt % of a neutralizer; and
the balance of water;
wherein the neutralizer has a structure represented by the general formula:

CₐH_{b}N_{c}O_{d}, where a≥6, b≥15, 1≤c<3, and 1≤d<6.

The individual components that may be comprised in the gel hand sanitizer according to the present invention and the method for preparing the gel hand sanitizer are described in detail below.

### Monohydric Alcohol

In some embodiments, the gel hand sanitizer may include a monohydric alcohol. The main role of the monohydric alcohol is to serve as a solvent and a disinfectant. The monohydric alcohol and the hydrogen peroxide mentioned below can act synergistically to improve fungi and virus killing effects of the gel hand sanitizer. There is no particular limitation on the specific type of the monohydric alcohol that can be used in the present invention, provided that the monohydric alcohol can provide proper functions of solvent and disinfectant. In some embodiments, the monohydric alcohol is one or more selected from the group consisting of ethanol, n-propanol, and isopropanol. The content of the monohydric alcohol is 75-85 wt % based on the total weight of the gel hand sanitizer. Commercially available products of the monohydric alcohol that can be used in the present invention include, for example, ethanol (product name: ethanol) produced by Sinopharm Chemical Reagent Co., Ltd.

### Hydrogen Peroxide

In some embodiments, the gel hand sanitizer may include hydrogen peroxide. The hydrogen peroxide acts as a disinfectant (acts to kill fungi and viruses). The hydrogen peroxide and the aforementioned monohydric alcohol can act synergistically to improve the fungi and virus killing effects of the gel hand sanitizer. The content of the hydrogen peroxide is 0.05-1 wt % based on the total weight of the gel hand sanitizer. In some embodiments, the content of the hydrogen peroxide is 0.05-0.5 wt % based on the total weight of the gel hand sanitizer. Commercially available products of the hydrogen peroxide that can be used in the present invention include, for example, hydrogen peroxide (product name: hydrogen peroxide) produced by Sinopharm Chemical Reagent Co., Ltd.

### Thickener

In some embodiments, the gel hand sanitizer according to the present invention may include a thickener. The thickener can enable the hand sanitizer of the present invention to form a gel system. In some embodiments, the thickener is selected from carbomer-based thickeners. In some embodiments, the carbomer-based thickener is a cross-linked copolymer of an acrylate and a C10-30 alkyl acrylate. In some embodiments, the viscosity of the carbomer-based thickener is 20000-90000 mPa·s. The viscosity of the carbomer-based thickener in the present invention is characterized by the viscosity of a 0.5 wt % aqueous dispersion obtained by adding the carbomer-based thickener to water to obtain a 0.5 wt % aqueous dispersion, then conducting a viscosity test on the resulting 0.5 wt % aqueous dispersion. For purposes of this patent application, viscosity is determined in accordance with the test method of the examples section. The content of the thickener is 0.1-2 wt % based on the total weight of the gel hand sanitizer. In some embodiments, the content of the thickener is 0.1-1 wt % based on the total weight of the gel hand sanitizer. Commercially available products of the thickener that can be used in the present invention are, for example, U20 (product name: U20) and ETD2020 (product name: ETD2020) produced by The Lubrizol Corporation.

### Neutralizer

In some embodiments, the gel hand sanitizer may include a neutralizer. The neutralizer can stabilize the hydrogen peroxide to ensure virus killing effects of the gel hand sanitizer. The neutralizer has a structure represented by the general formula: CₐH_{b}N_{c}O_{d}, where a≥6, b≥15, 1≤c<3, and 1≤d<6. In some embodiments, 6≤a<30, 15≤b<40, 1≤c<3, and 1≤d≤4. In some embodiments, 6≤a<15, 15≤b<35, 1≤c<3, and 1≤d≤4. In some embodiments, the neutralizer is one or more selected from the group consisting of triethanolamine, tetrahydroxypropylethylenediamine, and dimethylaminomethylpropanol. The content of the neutralizer is 0.01-1 wt % based on the total weight of the gel hand sanitizer. In some embodiments, the content of the neutralizer is 0.01-0.1 wt % based on the total weight of the gel hand sanitizer. Commercially available products of the neutralizer that can be used in the present invention are, for example, dimethylaminomethylpropanol (product name: DMAMP) produced by Angus Chemical Company and tetrahydroxyethylenediamine (product name: Quadrol L) produced by BASF Company.

### Other Components

The gel hand sanitizer according to the present invention may further optionally include one or more other components to achieve different functions. In some embodiments, the gel hand sanitizer further includes one or more selected from the group consisting of caprylyl glycol, a moisturizer, and the like. The content of the caprylyl glycol that can be used in the present invention is not particularly limited, and the addition of the caprylyl glycol can further improve the fungi killing (especially the killing of Candida albicans) properties of the gel hand sanitizer. The specific type and content of the moisturizer that can be used in the present invention are not particularly limited, and can be appropriately selected and added without affecting the antibacterial and antiviral effects etc. of the gel hand sanitizer according to the present invention. In some embodiments, the moisturizer is one or more selected from glycerin and a ceramide.

### Water

In the gel hand sanitizer according to the present invention, water is a solvent in the gel hand sanitizer. The specific content of water is not particularly limited provided that the monohydric alcohol, the hydrogen peroxide, the thickener, the neutralizer, and the optional other components mentioned above can be sufficiently dissolved or dispersed therein and the gel hand sanitizer can be adjusted thereby to a degree suitable for hand washing application in a gel state.

### Method for Preparing Gel Hand Sanitizer

The specific method for preparing a gel hand sanitizer according to the present invention is not particularly limited, and a mixing method commonly used in the art can be adopted. Specifically, the components of the gel hand sanitizer described above may be dissolved or dispersed together in the solvent according to the specific amounts thereof.

For the description of each component of the gel hand sanitizer, refer to the “gel hand sanitizer” section of this description for details.

The following detailed description is intended to illustrate the present disclosure by way of example rather than limitation.

Embodiment 1 is a gel hand sanitizer, including, based on the total weight thereof:
75-85 wt % of a monohydric alcohol;
0.05-1 wt % of hydrogen peroxide;
0.1-2 wt % of a thickener;
0.01-1 wt % of a neutralizer; and
the balance of water;
wherein the neutralizer has a structure represented by the general formula:

CₐH_{b}N_{c}O_{d}, where a≥6, b≥15, 1≤c<3, and 1≤d<6.

Embodiment 2 is the gel hand sanitizer according to Embodiment 1, wherein the monohydric alcohol is one or more selected from the group consisting of ethanol, n-propanol, and isopropanol.

Embodiment 3 is the gel hand sanitizer according to the Embodiment 1, wherein the thickener is selected from carbomer-based thickeners.

Embodiment 4 is the gel hand sanitizer according to Embodiment 3, wherein the carbomer-based thickener is a cross-linked copolymer of an acrylate and a C10-30 alkyl acrylate.

Embodiment 5 is the gel hand sanitizer according to Embodiment 3, wherein the viscosity of the carbomer-based thickener is 20000-90000 mPa·s.

Embodiment 6 is the gel hand sanitizer according to Embodiment 1, wherein the neutralizer is one or more selected from the group consisting of triethanolamine, tetrahydroxypropylethylenediamine, and dimethylaminomethylpropanol.

Embodiment 7 is the gel hand sanitizer according to Embodiment 1, wherein the gel hand sanitizer further includes caprylyl glycol.

Embodiment 8 is the gel hand sanitizer according to Embodiment 1, wherein the gel hand sanitizer further includes a moisturizer.

Embodiment 9 is the gel hand sanitizer according to Embodiment 8, wherein the moisturizer is selected from glycerin and/or a ceramide.

Embodiment 10 is a method for preparing a gel hand sanitizer, including mixing the components of the gel hand sanitizer according to any of Embodiments 1-9.

The present invention will be described in more detail below with reference to examples. It should be pointed out that these descriptions and examples are for the purpose of facilitating the understanding of the present invention, rather than limiting the present invention. The scope of protection of the present invention is subject to the appended claims.

### EXAMPLES

In the present invention, unless otherwise indicated, reagents used are all commercially available products, which are used directly without further purification.

### Test Methods

In the present disclosure, the gel hand sanitizers obtained in the following examples and comparative examples were tested for stability, moisture retention, fungi killing properties, and virus killing properties. The specific test methods are described below.

### Stability

The stability of the gel hand sanitizer was evaluated according to a reduction rate (%) of the content of hydrogen peroxide. After completion of formulation of a gel hand sanitizer sample, the sample was sealed and placed at room temperature for 1 day, and then tested for the content of hydrogen peroxide. The sample was then re-sealed and placed in a 37° C. oven for 90 days. The sample was taken out, and was tested for the content of hydrogen peroxide again when the temperature thereof returned to room temperature. The reduction rate between the two measurements of the content of hydrogen peroxide was calculated and recorded. If the reduction rate of hydrogen peroxide was less than or equal to 2%, the stability of the gel hand sanitizer was excellent; if the reduction rate of hydrogen peroxide was greater than 2% and less than or equal to 10%, the stability of the gel hand sanitizer was qualified; if the reduction rate of hydrogen peroxide was greater than or equal to 10%, the stability of the gel hand sanitizer was unqualified.

The content of hydrogen peroxide was tested according to Item 2.2.1.2.4 of the Technical Standard For Disinfection 2002.

2.2.1.2.4 Determination of Content of Hydrogen Peroxide (H₂O₂)

(1) Solutions of 2 mol/L sulfuric acid and 100 g/L manganese sulfate were prepared. A 0.02 mol/L potassium permanganate titration solution was additionally prepared and calibrated (see 2.2.1.3.3).

(2) An appropriate amount of the sample was precisely sucked to make it equivalent to about 0.3 g of hydrogen peroxide, and was diluted to a mark with distilled water in a 100-ml volumetric flask, and mixed well.

(3) 10.0 ml of the hydrogen peroxide diluent was taken and put in a 100-ml iodine flask, and 20 ml of 2 mol/L sulfuric acid and 3 drops of 100 g/L manganese sulfate were added and shaken well. The solution was titrated with the 0.02 mol/L potassium permanganate titration solution (in a 25 ml burette) until the solution turned pink, and the amount of the potassium permanganate titration solution used was recorded. The test was repeated twice, and the average value of the two measurements was taken for the following calculation.

(4) Since 1 ml of a 1 mol/L potassium permanganate titration solution is equivalent to 0.08505 g of hydrogen peroxide, the content of hydrogen peroxide could be calculated according to the following formula:

$$X ( g / L ) = \frac{c \times {V}_{pp} \times 0.08505}{V} \times 1000$$

In the formula, X was the content of hydrogen peroxide, in g/L; c was the concentration of the potassium permanganate titration solution, in mol/L; Vₚₚ was the volume of the potassium permanganate titration solution, in ml; and V was the volume of the hydrogen peroxide sample liquid in an iodine flask, in ml.

### Moisture Retention

### Preparation Before the Test:

Use of any product (cosmetics or medicine) was not allowed on a test site 2-3 days before the test, and contact with water was not allowed for 1 h to 3 h. Before the test, the inner forearms of both arms of each subject were required to be cleaned. The cleaning method was to wipe clean with a dry facial tissue. Before the formal test, the subject should sit still in a room that meets standards for at least 20 minutes, without drinking water and beverages, with the forearms exposed, placed in a test state, and kept relaxed.

### Test Apparatus:

Corneometer CM 825

### Test Method:

1. A product application area and a blank control area should be randomly distributed in marked areas of the left and right arms to ensure that the positions of all product and blank areas were statistically balanced.

2. A test sample was applied at a dosage of (2.0±0.1) mg/cm² in a single time, and the sample was evenly applied on the test area with a latex finger cot, and an actual applied amount was recorded.

3. After the instrument was adjusted according to the instruction manual of the Corneomter tester, the product area and the control area were measured, with each area being tested in parallel determination at least three times. The initial value of each test area was first measured (before use of the sample), and then the moisture contents of skin of the test area and the control area were measured after use of the hand sanitizer. The set times were kept the same.

Three test results of the test area were averaged to obtain the test result of the moisture content of skin of the test area. If the test result was less than or equal to 20 g/hm², the test result of moisture retention was qualified; if the test result was greater than 20 g/hm², the test result of moisture retention was unqualified.

### Fungi Killing Properties

A fungi killing properties test tested the gel hand sanitizers obtained in the following examples and comparative examples for fungi killing properties on Candida albicans in accordance with the fungi killing test recorded in Section 2.1.1.9 of the Technical Standard

### For Disinfection 2002.

When the killing rate of Candida albicans was greater than or equal to 99.990% and less than 99.999%, the fungi killing properties were qualified; when the killing rate of Candida albicans was greater than or equal to 99.999%, the fungi killing properties were excellent; when the killing rate of Candida albicans was less than 99.990%, the fungi killing properties were unqualified.

### Virus Killing Properties

A virus killing properties test tested the gel hand sanitizers obtained in the following examples and comparative examples for poliovirus killing properties in accordance with the poliovirus inactivation test recorded in Section 2.1.1.10.7 of the Technical Standard For Disinfection 2002.

If the mean log inactivation value was ≥3.00 and <4.00, the sample could be determined to be qualified in poliovirus killing properties; if the mean log inactivation value was ≥4.00, the sample could be determined to be excellent in poliovirus killing properties; if the mean log inactivation value was <3.00, the sample could be determined to be unqualified in poliovirus killing properties.

### Viscosity Test

The viscosity of the gel hand sanitizers obtained in the following examples and comparative examples was tested in accordance with a Brookfield viscosity test method.

The amount of the gel hand sanitizer should be sufficient to submerge ½ or more of a rotor at room temperature. First, a corresponding rotor and speed should be selected with reference to the following table. A cement spatula could be used to stir in the solution to estimate the viscosity thereof.

| | |
|---|---|
| Speed | Rotor number |

| (RPM) | 1 | 2 | 3 | 4 |
|---|---|---|---|---|
| 6 | 1,000 | 5,000 | 20,000 | 100,000 |
| 12 | 500 | 2,500 | 10,000 | 50,000 |
| 30 | 200 | 1,000 | 4,000 | 20,000 |
| 60 | 100 | 500 | 2,000 | 10,000 |

During testing of the viscosity, the rotor was inserted into the solution under test until a groove on the rotor was submerged in the sample. For a disc-type rotor, the rotor was slightly tilted to avoid trapping air bubbles. When the rotor was installed, a viscometer connector was held by one hand and gently lifted, and the rotor was turned to the left by the other hand to tighten up the rotor. The rotor was then inserted into the sample to an appropriate depth. Subsequently, to measure the viscosity, an appropriate rotor and rotation speed should be selected, and a value was read after the value was stable. To obtain the maximum measurement accuracy, % (inductive torque) readings should be greater than 10%. To replace the rotor or sample, a MOTORON/OFF key was pressed to turn off the motor. The rotor should be cleaned up. After the reading of the viscosity value was stabilized, the result displayed by the instrument was recorded.

When the viscosity was greater than or equal to 5000 cps, the viscosity test result was qualified; and when the viscosity was less than 5000 cps, the viscosity test result was unqualified.

Reagents used in the following examples and comparative examples were specifically listed in Table 1 below:

TABLE 1

| Function | Product name | Chemical name | Supplier |
|---|---|---|---|
| Monohydric | Ethanol | Ethanol | Sinopharm Chemical |
| alcohol | | | Reagent Co., Ltd. |
| | n-Propanol | n-Propanol | Sinopharm Chemical |
| | | | Reagent Co., Ltd. |
| | Isopropanol | Isopropanol | Sinopharm Chemical |
| | | | Reagent Co., Ltd. |
| Hydrogen | Hydrogen | 30% aqueous hydrogen | Sinopharm Chemical |
| peroxide | peroxide | peroxide solution | Reagent Co., Ltd. |
| Thickener | U20 | Modified acrylic | Lubrizol China Co., |
| | | polymer | Ltd. |
| | ETD 2020 | Modified acrylic | Lubrizol China Co., |
| | | polymer | Ltd. |
| | Methylcellulose | Methylcellulose | Sinopharm Chemical |
| | | | Reagent Co., Ltd. |
| Neutralizer | TEOA | Triethanolamine | Sinopharm Chemical |
| | | | Reagent Co., Ltd. |
| | Quadrol L | Tetrahydroxypropyl- | BASF China Co., Ltd. |
| | | ethylenediamine | |
| | DMAMP | Dimethylaminomethyl | Angus Chemical |
| | | propanol | China Co., Ltd. |
| | AMP PC2000 | Aminomethylpropanol | Angus Chemical |
| | | | China Co., Ltd. |
| Caprylyl | Caprylyl glycol | Caprylyl glycol | Sinopharm Chemical |
| glycol | | | Reagent Co., Ltd. |
| Moisturizer | Ceramide | Ceramide | Evonik China Co., Ltd. |
| | Glycerin | Glycerin | Sinopharm Chemical |
| | | | Reagent Co., Ltd. |

### Example 1 (E1)

At a temperature of 25° C. to 30° C., 75 g of ethanol, 0.1 g of hydrogen peroxide, 0.5 g of a thickener U20, 0.04 g of a neutralizer TEOA, 1 g of caprylyl glycol, 0.5 g of glycerol, and 1 g of ceramide were dissolved in 21.86 g of deionized water, and stirred well to prepare a gel hand sanitizer 1.

The resulting gel hand sanitizer 1 was tested with respect to stability, moisture retention, fungi killing properties, virus killing properties, and viscosity according to the test methods described above, and the specific test results were respectively shown in Table 5 below.

### Examples 2-20 (E2-E20) and Comparative Examples 1-7 (C1-C7)

Examples 2-20 (E2-E20) and Comparative Examples 1-7 (C1-C7) were prepared in a similar manner to that in Example 1, except that the amounts of the components were changed according to the proportions shown in Tables 2, 3, and 4 below to obtain gel hand sanitizers.

The gel hand sanitizers obtained in Examples 2-20 (E2-E20) and Comparative Examples 1-7 (C1-C7) were tested with respect to stability, moisture retention, fungi killing properties, virus killing properties, and viscosity respectively according to the test methods described above, and the specific test results were respectively shown in Table 5 below.

TABLE 2
Proportions of components of gel hand sanitizers prepared in Examples 1-10 (E1-E10)

| | | E1 | E2 | E3 | E4 | E5 | E6 | E7 | E8 | E9 | E10 |
|---|---|---|---|---|---|---|---|---|---|---|---|
| Function | Name | % w/w | % w/w | % w/w | % w/w | % w/w | % w/w | % w/w | % w/w | % w/w | % w/w |
| Monohydric alcohol | Ethanol | 75 | 85 | 80 | 80 | 80 | 80 | 80 | 80 | 80 | 80 |
| | n-Propanol | 0 | 0 | 0 | 0 | 0 | 0 | 0 | 0 | 0 | 0 |
| | Isopropanol | 0 | 0 | 0 | 0 | 0 | 0 | 0 | 0 | 0 | 0 |
| Hydrogen peroxide | Hydrogen peroxide | 0.1 | 0.1 | 0.05 | 1 | 0.1 | 0.1 | 0.1 | 0.1 | 0.1 | 0.1 |
| Thickener | U20 | 0.5 | 0.5 | 0.5 | 0.5 | 0.1 | 2 | 0.5 | 0.5 | 0.5 | 0.5 |
| | ETD 2020 | 0 | 0 | 0 | 0 | 0 | 0 | 0 | 0 | 0 | 0 |
| | Methylcellulose | 0 | 0 | 0 | 0 | 0 | 0 | 0 | 0 | 0 | 0 |
| Neutralizer | TEOA | 0.04 | 0.04 | 0.04 | 0.04 | 0.08 | 0.04 | 0.01 | 1 | 0 | 0 |
| | Quadrol L | 0 | 0 | 0 | 0 | 0 | 0 | 0 | 0 | 0.01 | 1 |
| | DMAMP | 0 | 0 | 0 | 0 | 0 | 0 | 0 | 0 | 0 | 0 |
| | AMP PC2000 | 0 | 0 | 0 | 0 | 0 | 0 | 0 | 0 | 0 | 0 |
| Caprylyl Glycol | Caprylyl Glycol | 1 | 1 | 1 | 1 | 1 | 1 | 1 | 1 | 1 | 1 |
| Moisturizer | Glycerin | 0.5 | 0.5 | 0.5 | 0.5 | 0.5 | 0.5 | 0.5 | 0.5 | 0.5 | 0.5 |
| Moisturizer | Ceramide | 1 | 1 | 1 | 1 | 1 | 1 | 1 | 1 | 1 | 1 |
| Water | Water | 21.86 | 11.86 | 16.91 | 15.96 | 17.22 | 15.36 | 16.89 | 15.9 | 16.89 | 15.9 |
| Total | 100 | 100 | 100 | 100 | 100 | 100 | 100 | 100 | 100 | 100 | 100 |

TABLE 3
Proportions of components of gel hand sanitizers prepared in Examples 11-20 (E11-E20)

| | | E11 | E12 | E13 | E14 | E15 | E16 | E17 | E18 | E19 | E20 |
|---|---|---|---|---|---|---|---|---|---|---|---|
| Function | Name | % w/w | % w/w | % w/w | % w/w | % w/w | % w/w | % w/w | % w/w | % w/w | % w/w |
| Monohydric alcohol | Ethanol | 80 | 80 | 75 | 75 | 75 | 75 | 75 | 75 | 0 | 0 |
| | n-Propanol | 0 | 0 | 0 | 0 | 0 | 0 | 0 | 0 | 80 | 0 |
| | Isopropanol | 0 | 0 | 0 | 0 | 0 | 0 | 0 | 0 | 0 | 80 |
| Hydrogen peroxide | Hydrogen peroxide | 0.1 | 0.1 | 0.05 | 0.1 | 0.1 | 0.1 | 0.1 | 0.1 | 0.1 | 0.1 |
| Thickener | U20 | 0.5 | 0.5 | 0.5 | 0.5 | 0.5 | 0.5 | 0 | 0.5 | 0.5 | 0.5 |
| | ETD 2020 | 0 | 0 | 0 | 0 | 0 | 0 | 0.5 | 0 | 0 | 0 |
| | Methylcellulose | 0 | 0 | 0 | 0 | 0 | 0 | 0 | 0 | 0 | 0 |
| Neutralizer | TEOA | 0.04 | 0.04 | 0.04 | 0.04 | 0.04 | 0.04 | 0.04 | 0 | 0.05 | 0.05 |
| | Quadrol L | 0 | 0 | 0 | 0 | 0 | 0 | 0 | 0 | 0 | 0 |
| | DMAMP | 0 | 0 | 0 | 0 | 0 | 0 | 0 | 0.05 | 0 | 0 |
| | AMP PC2000 | 0 | 0 | 0 | 0 | 0 | 0 | 0 | 0 | 0 | 0 |
| Caprylyl glycol | Caprylyl glycol | 0.5 | 5 | 0 | 1 | 1 | 1 | 1 | 1 | 1 | 1 |
| Moisturizer | Glycerin | 0.5 | 0.5 | 0.5 | 1.5 | 0 | 0 | 0.5 | 0.5 | 0.5 | 0.5 |
| Moisturizer | Ceramide | 1 | 1 | 1 | 0 | 1 | 1 | 0.5 | 0.5 | 0.5 | 0.5 |
| Water | Water | 17.36 | 12.86 | 22.91 | 21.86 | 22.36 | 22.36 | 22.36 | 22.35 | 17.35 | 17.35 |
| Total | 100 | 100 | 100 | 100 | 100 | 100 | 100 | 100 | 100 | 100 | 100 |

TABLE 4
Proportions of components of gel hand sanitizers prepared in Comparative Examples 1-7 (C1-C7)

| | | C1 | C2 | C3 | C4 | C5 | C6 | C7 |
|---|---|---|---|---|---|---|---|---|
| Function | Name | % w/w | % w/w | % w/w | % w/w | % w/w | % w/w | % w/w |
| Monohydric alcohol | Ethanol | 65 | 75 | 80 | 75 | 80 | 80 | 85 |
| | n-Propanol | 0 | 0 | 0 | 0 | 0 | 0 | 0 |
| | Isopropanol | 0 | 0 | 0 | 0 | 0 | 0 | 0 |
| Hydrogen peroxide | Hydrogen peroxide | 0.1 | 0 | 0.1 | 0.1 | 0.1 | 0.1 | 0.1 |
| Thickener | U20 | 0.5 | 0.5 | 0.01 | 0 | 0.5 | 0.5 | 0.5 |
| | ETD 2020 | 0 | 0 | 0 | 0 | 0 | 0 | 0 |
| | Methylcellulose | 0 | 0 | 0 | 0.5 | 0 | 0 | 0 |
| Neutralizer | TEOA | 0.04 | 0.04 | 0.04 | 0 | 0 | 0.001 | 0 |
| | Quadrol L | 0 | 0 | 0 | 0 | 0.001 | 0 | 0 |
| | DMAMP | 0 | 0 | 0 | 0 | 0 | 0 | 0 |
| | AMP PC2000 | 0 | 0 | 0 | 0 | 0 | 0 | 0.04 |
| Caprylyl glycol | Caprylyl glycol | 1 | 1 | 1 | 1 | 1 | 1 | 1 |
| Moisturizer | Glycerin | 0.5 | 0 | 0.5 | 0 | 0.5 | 0.5 | 0.5 |
| Moisturizer | Ceramide | 1 | 1 | 1 | 1 | 1 | 1 | 1 |
| Water | Water | 31.86 | 22.46 | 17.35 | 22.4 | 16.899 | 16.899 | 11.86 |
| Total | 100 | 100 | 100 | 100 | 100 | 100 | 100 | 100 |

TABLE 5
Stability, moisture retention, fungi killing properties, virus killing
properties, and viscosity of the test results of gel hand sanitizers
prepared in Examples 1-20 (E1-E20) and Comparative Examples 1-7 (C1-C7)

| | | Virus killing | | | |
|---|---|---|---|---|---|
| | Reduction | properties | | | |
| | rate (%) | (mean log | | Moisture | |
| | of hydrogen | inactivation | Fungi killing | retention | Viscosity |
| | peroxide | value) | properties (%) | (g/hm²) | (cps) |
| Example 1 | 3.00 | 3.85 | >99.999 | 7.9 | 8300 |
| Example 2 | 3.00 | 4.05 | >99.999 | 13.4 | 8112 |
| Example 3 | 1.00 | 3.1 | >99.999 | 8.2 | 8367 |
| Example 4 | 4.00 | 4.56 | >99.999 | 8.3 | 8299 |
| Example 5 | 1.00 | 4.1 | >99.999 | 8.3 | 6214 |
| Example 6 | 1.00 | 4.01 | >99.999 | 8.2 | 14502 |
| Example 7 | 2.00 | 4.18 | >99.999 | 8.2 | 6200 |
| Example 8 | 1.00 | 3.87 | >99.999 | 8.3 | 23900 |
| Example 9 | 1.00 | 4.01 | >99.999 | 8.3 | 5899 |
| Example 10 | 4.00 | 4.02 | >99.999 | 8.7 | 15675 |
| Example 11 | 3.00 | 4.01 | >99.999 | 10.9 | 8379 |
| Example 12 | 2.00 | 4.21 | >99.999 | 6.1 | 8276 |
| Example 13 | 4.00 | 3.8 | >99.990 | 9.7 | 8334 |
| Example 14 | 2.00 | 3.78 | >99.999 | 12.2 | 7800 |
| Example 15 | 3.50 | 3.8 | >99.999 | 11.4 | 7910 |
| Example 16 | 3.00 | 3.8 | >99.999 | 15.9 | 7830 |
| Example 17 | 3.50 | 3.6 | >99.999 | 8.6 | 6700 |
| Example 18 | 4.00 | 3.7 | >99.999 | 8.6 | 6800 |
| Example 19 | 3.60 | 3.8 | >99.999 | 7.3 | 8800 |
| Example 20 | 3.80 | 3.8 | >99.999 | 7.6 | 8750 |
| Comparative | / | 2.45 | / | / | / |
| Example 1 | | | | | |
| Comparative | / | 1.81 | / | / | / |
| Example 2 | | | | | |
| Comparative | / | / | / | / | 368 |
| Example 3 | | | | | |
| Comparative | / | / | / | / | 2360 |
| Example 4 | | | | | |
| Comparative | / | / | / | / | 1879 |
| Example 5 | | | | | |
| Comparative | / | / | / | / | 2100 |
| Example 6 | | | | | |
| Comparative | 11.00 | / | / | / | 3800 |
| Example 7 | | | | | |

As can be seen from the test results shown in Table 5 above, when the specific amounts of the monoalcohol, the hydrogen peroxide, the thickener, the specific neutralizer, and water were selected within the scopes of the present invention to prepare gel hand sanitizers, the resulting gel hand sanitizers had good stability and moisture retention, and had excellent killing effects on Candida albicans and polioviruses at the same time.

By comparing Example 1 with Comparative Example 1, it can be seen that when the content (65 wt %) of the monohydric alcohol in the gel hand sanitizer was less than the lower limit (75 wt %) specified in the present invention, the resulting gel hand sanitizer had poor virus killing effects.

By comparing Example 15 with Comparative Example 2, it can be seen that when the gel hand sanitizer did not contain hydrogen peroxide, the resulting gel hand sanitizer had poor virus killing effects.

By comparing Example 6 with Comparative Example 3, it can be seen that when the content (0.01 wt %) of the thickener in the gel hand sanitizer was less than the lower limit (0.1 wt %) specified in the present invention, the viscosity of the resulting hand sanitizer was overly low to form a gel system.

By comparing Example 1 with Comparative Example 4, it can be seen that when the thickener in the gel hand sanitizer was a cellulose-based thickener (not a carbomer-based thickener), the viscosity of the resulting hand sanitizer was overly low to form a gel system.

By comparing Example 9 with Comparative Example 5 and comparing Example 7 with Comparative Example 6, it can be seen that when the content (0.001 wt %) of the neutralizer in the gel hand sanitizer was less than the lower limit (0.01 wt %) specified in the present invention, the viscosity of the resulting hand sanitizer was overly low to form a gel system.

By comparing Example 2 with Comparative Example 7, it can be seen that when the neutralizer in the gel hand sanitizer was aminomethylpropanol (which did not have the general formula CₐH_{b}N_{c}O_{d} (where a≥6, b≥15, 1≤c<3, and 1≤d<6)), the content of hydrogen peroxide in the resulting gel hand sanitizer was greatly reduced (reduced by 11%); therefore, the resulting gel hand sanitizer had poor stability, which could not form a gel system.

Although specific embodiments have been shown and described in the present invention, a person skilled in the art would understand that various alternative and/or equivalent embodiments may be used to substitute the specific embodiments shown and described without departing from the scope of the present invention. The present application is intended to encompass any adaptations or variations of the specific embodiments discussed in the present invention. Accordingly, the present invention is subject only to the claims and equivalents thereof.

A person skilled in the art should appreciate that various modifications and changes could be made without departing from the scope of the present invention. Such modifications and changes are intended to fall within the scope of the present invention as defined by the appended claims.

1. A gel hand sanitizer, comprising, based on a total weight of the gel hand sanitizer:
75-85 weight percent of a monohydric alcohol;
0.05-1 weight percent of hydrogen peroxide;
0.1-2 weight percent of a thickener;
0.01-1 weight percent of a neutralizer; and
a remaining weight percent of water;
wherein the neutralizer has a structure represented by a general formula: CₐH_{b}N_{c}O_{d}, where a≥6, b≥15, 1≤c<3, and 1≤d<6.

2. The gel hand sanitizer according to claim 1, wherein the monohydric alcohol is selected from the group consisting of ethanol, n-propanol, and isopropanol.

3. The gel hand sanitizer according to claim 1, wherein the thickener comprises a carbomer-based thickener.

4. The gel hand sanitizer according to claim 3, wherein the carbomer-based thickener is a cross-linked copolymer of an acrylate and a C10-30 alkyl acrylate.

5. The gel hand sanitizer according to claim 3, wherein a viscosity of the carbomer-based thickener is 20000-90000 millipascal-second.

6. The gel hand sanitizer according to claim 1, wherein the neutralizer is selected from the group consisting of triethanolamine, tetrahydroxypropylethylenediamine, and dimethylaminomethylpropanol.

7. The gel hand sanitizer according to claim 1, further comprising caprylyl glycol.

8. The gel hand sanitizer according to claim 1, further comprising a moisturizer.

9. The gel hand sanitizer according to claim 8, wherein the moisturizer is selected from the group consisting of glycerin and a ceramide.

10. A method for preparing a gel hand sanitizer, comprising mixing the components of the gel hand sanitizer according to claim 1.
